(19) United States
(12) Patent Application Publication
(10) Pub. No.: US 2010/0168701 A1
(43) Pub. Date: Jul. 1, 2010

(54) Absorbent Articles With Primary and Secondary Indicia

(76) Inventors: Mattias Schmidt, Idstein (DE); Miguel Alvaro Robles, Wyoming, OH (US); Donald Carroll Roe, Chester, OH (US); Michael I. Divo, Friedrichsdorf (DE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/646,430

(22) Filed: Dec. 23, 2009

Related U.S. Application Data
(60) Provisional application No. 61/141,573, filed on Dec. 30, 2008.

(51) Int. Cl. A61F 13/42

(52) U.S. Cl. 604/361

(57) ABSTRACT

An absorbent article comprises a visual fullness indicator along with primary visual indicia and a secondary visual indicium.

### CROSS REFERENCE TO RELATED APPLICATIONS

This application claims the benefit of U.S. provisional patent application 61/141,573 filed Dec. 30, 2008, the substance of which is hereby incorporated by reference.

### FIELD

In general, embodiments of the present disclosure relate to wetness indicating for absorbent articles. In particular, embodiments of the present disclosure relate to visual fullness indicating for absorbent articles.

### BACKGROUND

Absorbent articles can absorb liquid bodily exudates such as sweat, blood, urine, menses, etc. An absorbent article can include a wetness indicator. The wetness indicator can indicate the presence of a liquid bodily exudate in the article. Unfortunately, some wetness indicators for absorbent articles can be difficult to understand. If the signal from a wetness indicator is misunderstood then the absorbent article may be changed too soon. The wearer may underutilize the capacity of the article. If the signal from a wetness indicator is misunderstood then the absorbent article may be changed too late. The bodily exudates may exceed the capacity of the article resulting in leaks.

### BRIEF DESCRIPTION OF THE DRAWINGS

FIG. 1A illustrates a pant-type disposable wearable absorbent article with a visual fullness indicator as well as primary visual indicia, secondary visual indicia, and tertiary visual indicia in the front, according to embodiments of the present disclosure.

FIG. 1B illustrates a pant-type disposable wearable absorbent article with a visual fullness indicator as well as primary visual indicia, secondary visual indicia, and tertiary visual indicia in the back, according to embodiments of the present disclosure.

FIG. 1C illustrates a pant-type disposable wearable absorbent article with a number of visual fullness indicators as well as primary visual indicia, secondary visual indicia, and tertiary visual indicia, according to embodiments of the present disclosure.

FIG. 2A illustrates a front-fastenable disposable wearable absorbent article with a visual fullness indicator as well as primary visual indicia, secondary visual indicia, and tertiary visual indicia in the front, according to embodiments of the present disclosure.

FIG. 2B illustrates a front-fastenable disposable wearable absorbent article with a visual fullness indicator as well as primary visual indicia, secondary visual indicia, and tertiary visual indicia in the back, according to embodiments of the present disclosure.

FIG. 2C illustrates a front-fastenable disposable wearable absorbent article with a number of visual fullness indicators as well as primary visual indicia, secondary visual indicia, and tertiary visual indicia, according to embodiments of the present disclosure.

FIG. 3A illustrates a portion of an absorbent article with a visual fullness indicator, as well as primary visual indicia, secondary visual indicia, and tertiary visual indicia, according to embodiments of the present disclosure.

FIG. 3B illustrates a subsequent state of indication for the indicator and visual indicia of FIG. 3A, according to embodiments of the present disclosure.

FIG. 3C illustrates a subsequent state of indication for the indicator and visual indicia of FIG. 3B, according to embodiments of the present disclosure.

FIG. 3D illustrates a subsequent state of indication for the indicator and visual indicia of FIG. 3C, according to embodiments of the present disclosure.

FIG. 3E illustrates a subsequent state of indication for the indicator and visual indicia of FIG. 3D, according to embodiments of the present disclosure.

FIG. 3F illustrates a subsequent state of indication for the indicator and visual indicia of FIG. 3E, according to embodiments of the present disclosure.

FIG. 3G illustrates a subsequent state of indication for the indicator and visual indicia of FIG. 3F, according to embodiments of the present disclosure.

FIG. 4 illustrates a portion of an absorbent article with a visual fullness indicator, as well as primary visual indicia, secondary visual indicia, and tertiary visual indicia, wherein the visual indicia are spaced apart from each other, according to embodiments of the present disclosure.

### SUMMARY

The present disclosure includes absorbent articles with indicators that are easy to understand. The absorbent articles of the present disclosure are easy to understand because they have primary and secondary visual indicia.

As an example, an absorbent article can have a visual fullness indicator along with primary visual indicia and secondary visual indicia. The indicator can progressively change visual states as the article becomes filled with a liquid bodily exudate. The primary and secondary visual indicia can be positioned in the article to correlate with varying degrees of fullness.

First visual fullness indicator can begin to change visual states along a first portion of the indicator. Primary visual indicia positioned proximate to the first portion can indicate that the absorbent article is somewhat filled. Second, the visual fullness indicator can change visual states along a second portion of the indicator. Secondary visual indicia positioned proximate to the second portion can indicate that the absorbent article is approaching full. The combination of the visual fullness indicator along with the primary visual indicia and the secondary visual indicia can be easily understood as indicating degrees of fullness. Together, the combination of the visual fullness indicator along with primary and secondary visual indicia can be considered a visual fullness indicating system.

An absorbent article having primary visual indicia and secondary visual indicia along with a visual fullness indicator can help provide certainty about the fullness of the absorbent article. By knowing how full an article is, the article can be changed after the wearer has appropriately utilized the capacity of the article. Also, by knowing how full an article is, the article can be changed before it is likely to leak.

### DETAILED DESCRIPTION

The primary and secondary visual indicia of the present disclosure can be used with all kinds of absorbent articles. An absorbent article can absorb liquid bodily exudates such as sweat, blood, urine, menses, etc. An absorbent article can be a product or a material. Examples of absorbent articles include products and/or materials for sanitary protection, hygienic use, and/or wound care.

Some absorbent articles are disposable. A disposable absorbent article is configured to be partly or wholly disposed of after a single use. A disposable absorbent article is configured such that the soiled article, or a soiled portion of the article, is not intended to be restored and reused (e.g., not intended to be laundered). Examples of disposable absorbent articles include wound care products, such as bandages and dressings, as well as feminine care products, such as pads and liners. Disposable absorbent articles can use embodiments of the present disclosure.

Some absorbent articles are wearable. A wearable absorbent article is configured to be worn on or around a body of a wearer. Wearable absorbent articles can also be disposable. Examples of disposable wearable absorbent articles include disposable diapers and disposable incontinence undergarments. A disposable wearable absorbent article can receive and contain bodily exudates while being worn by a wearer. In some embodiments, a disposable wearable absorbent article can include a topsheet, an absorbent core, an outer cover, a waist opening, and leg openings. Disposable wearable absorbent articles can use embodiments of the present disclosure.

One kind of wetness indicator for an absorbent article is a visual fullness indicator. A wetness indicator is considered visual if it can indicate the presence of a liquid bodily exudate by its visual state. Throughout the present disclosure, unless otherwise stated, the presence of a liquid bodily exudate refers to the presence of a concentration of the liquid bodily exudate that is sufficient to cause a visual wetness indicator to change visual states. A wetness indicator is considered a fullness indicator if it can indicate the degree to which a liquid bodily exudate has filled an absorbent article. A visual fullness indicator can indicate the presence of a liquid bodily exudate by a wet edge that moves along the indicator such that the indicator progressively changes visual states. A visual fullness indicator can include one or more visual indicating areas. A visual indicating area is a defined continuous two-dimensional region, configured to indicate the presence of a liquid bodily exudate by its visual state. As examples, in various embodiments, an indicator can comprise a series of indicating areas or a pattern of indicating areas. A visual indicium is visual device that can be configured to visually indicate a particular degree of fullness.

The figures of the present disclosure are intended to illustrate elements, their parts, and their relationships, as described in the specification; the figures are not intended to illustrate any particular relative or absolute size or dimension, unless otherwise stated in the text.

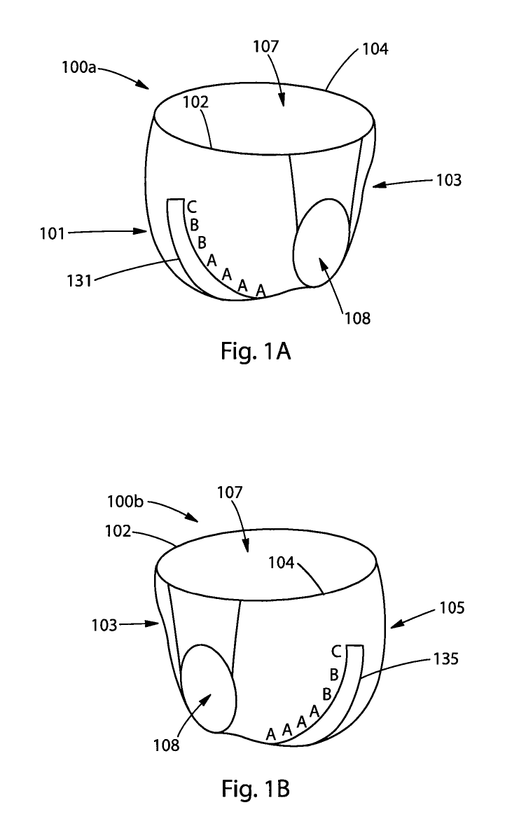

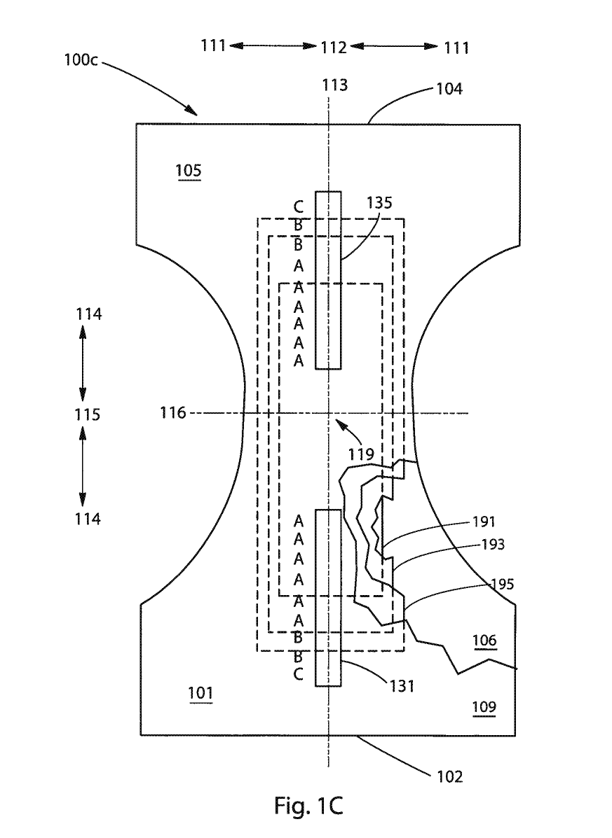

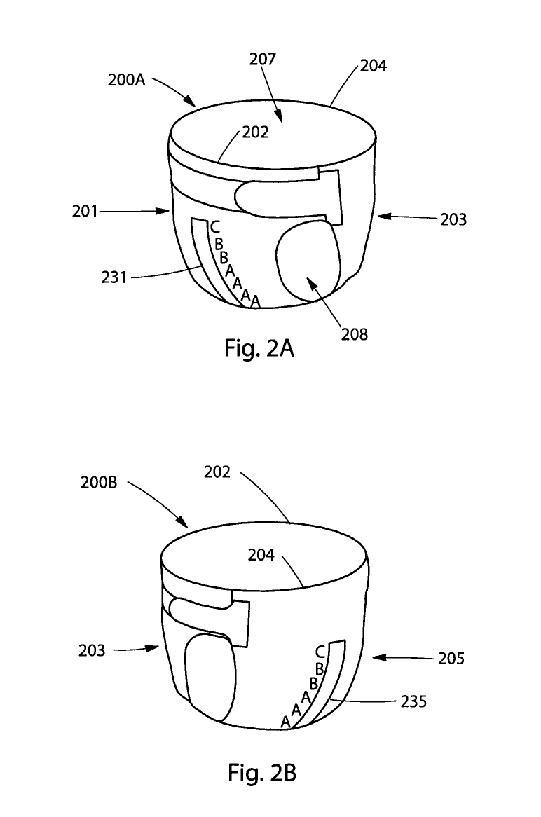

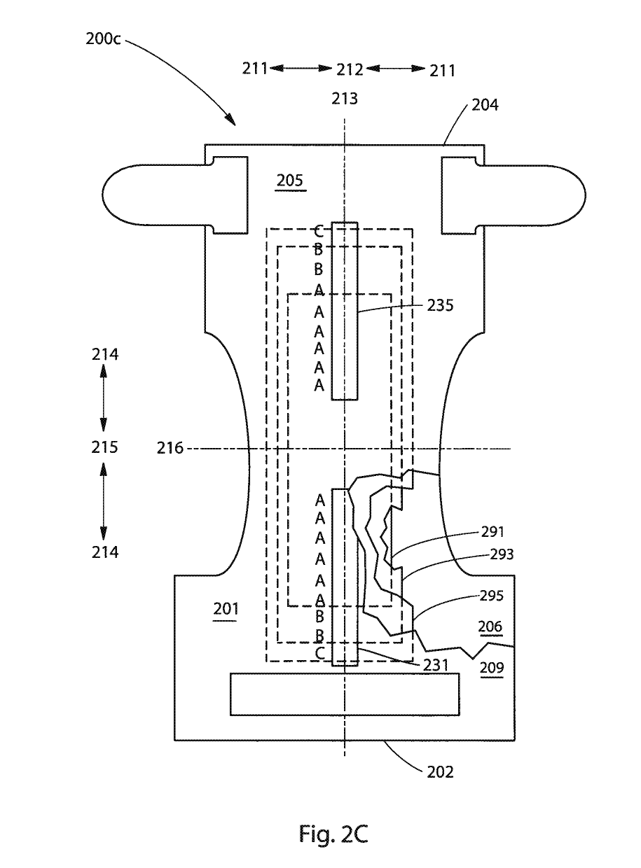

FIGS. 1A-2C illustrate various disposable wearable absorbent articles, each with one or more indicators along with primary and secondary visual indicia. For clarity, FIGS. 1A-2C do not illustrate all details of the indicators or of the disposable wearable absorbent articles. Each indicator in FIGS. 1A-2C can be any embodiment of an indicator of the present disclosure. Further, each primary visual indicium in FIGS. 1A-2C can be any embodiment of a primary visual indicium of the present disclosure, each secondary visual indicium in FIGS. 1A-2C can be any embodiment of a secondary visual indicium of the present disclosure, and each tertiary visual indicium in FIGS. 1A-2C can be any embodiment of a tertiary visual indicium of the present disclosure as described herein.

FIG. 1A illustrates an outside perspective view of a front 101 and a side 103 of a pant-type disposable wearable absorbent article 100A formed for wearing. The absorbent article 100A includes a front waist edge 102 and a back waist edge 104. The pant-type disposable wearable absorbent article 100A also includes a waist opening 107 and a leg opening 108. The absorbent article 100A includes a longitudinally oriented visual fullness indicator 131 disposed in the front 101. The absorbent article 100A further includes primary visual indicia represented by the letter A, secondary visual indicia represented by the letter B, and a tertiary visual indicium represented by the letter C, disposed in the front 101. Together, the visual fullness indicator 131 along with the primary, secondary, and tertiary visual indicia A, B, and C can be considered a visual fullness indicating system.

Throughout the present disclosure, a reference to a pant-type disposable wearable absorbent article can refer to an embodiment that is side-fastenable or to an embodiment without fasteners. A reference to a pant-type disposable wearable absorbent article can also refer to an article with preformed waist and/or leg openings or to an embodiment that is not preformed. Thus, each embodiment of an absorbent article of the present disclosure that is described as pant-type can be configured in any of these ways, as will be understood by one of ordinary skill in the art.

FIG. 1B illustrates an outside perspective view of a side 103 and a back 105 of a pant-type disposable wearable absorbent article 100B formed for wearing. The absorbent article 100B includes a front waist edge 102 and a back waist edge 104. The pant-type disposable wearable absorbent article 100B also includes a waist opening 107 and a leg opening 108. The absorbent article 100B includes a longitudinally oriented visual fullness indicator 135 disposed in the back 105. The absorbent article 100B further includes primary visual indicia represented by the letter A, secondary visual indicia represented by the letter B, and a tertiary visual indicium represented by the letter C, disposed in the back 105. Together, the visual fullness indicator 135 along with the primary, secondary, and tertiary visual indicia A, B, and C can be considered a visual fullness indicating system.

FIG. 1C illustrates an outside plan view of a pant-type disposable wearable absorbent article 100C laid out flat. The disposable wearable absorbent article 100C includes a front 101 and aback 105, which are separated by a lateral centerline 116. The absorbent article 100C also includes a front waist edge 102 and a back waist edge 104.

In FIG. 1C, a longitudinal centerline 113 and the lateral centerline 116 provide lines of reference for referring to relative locations of the disposable wearable absorbent article 100C. When a first location is nearer to the longitudinal centerline 113 than a second location, the first location can be considered laterally inboard 112 to the second location. Similarly, the second location can be considered laterally outboard 111 from the first location. When a third location is nearer to the lateral centerline 116 than a fourth location, the third location can be considered longitudinally inboard 115 to the fourth location. Also, the fourth location can be considered longitudinally outboard 114 from the third location.

A reference to an inboard location, without a lateral or longitudinal limitation, refers to a location of the disposable wearable absorbent article 100C that is laterally inboard and/or longitudinally inboard to another location. In the same way, a reference to an outboard location, without a lateral or longitudinal limitation, refers to a location of the disposable wearable absorbent article 100C that is laterally outboard and/or longitudinally outboard from another location.

Inboard and outboard can also be understood with reference to a center of a disposable wearable absorbent article. The longitudinal centerline 113 and the lateral centerline 116 cross at a center 119 of the disposable wearable absorbent article 100C. When one location is nearer to the center 119 than another location, the one location can be considered inboard to the other location. The one location can be inboard laterally, or longitudinally, or both laterally and longitudinally. The other location can be considered outboard from the one location. The other location can be outboard laterally, or longitudinally, or both laterally and longitudinally.

FIG. 1C includes arrows indicating relative directions for laterally outboard 111, laterally inboard 112, longitudinally outboard 114, and longitudinally inboard 115, each with respect to the disposable wearable absorbent article 100C. Throughout the present disclosure, a reference to a longitudinal dimension, measurement, line, or direction refers to a dimension, measurement, line, or direction that is substantially or completely parallel to the longitudinal centerline 113 and a reference to a lateral dimension, measurement, line, or direction refers to a dimension, measurement, line, or direction that is substantially or completely parallel to the lateral centerline 116. The terminology for describing relative locations, as discussed above, is used for disposable wearable absorbent articles throughout the present disclosure. This terminology can also be similarly applied to various other absorbent articles, as will be understood by one of ordinary skill in the art.

The disposable wearable absorbent article 100C includes a topsheet 106, an outer cover 109, an acquisition layer 191, a distribution layer 193, and an absorbent core 195. A portion of the outer cover 109 is shown as broken to illustrate a portion of the topsheet 106 and a portion of the absorbent core 195. A portion of the absorbent core 195 is shown as broken to illustrate a portion of the distribution layer 193. A portion of the distribution layer 193 is shown as broken to illustrate a portion of the acquisition layer 191.

The disposable wearable absorbent article 100C includes a number of indicators in various exemplary locations and orientations. The disposable wearable absorbent article 100C includes a longitudinally oriented visual fullness indicator 131, along the longitudinal centerline 113 in the front 101. The front 101 also includes primary visual indicia represented by the letter A, secondary visual indicia represented by the letter B, and a tertiary visual indicium represented by the letter C. Together, the visual fullness indicator 131 along with the primary, secondary, and tertiary visual indicia A, B, and C can be considered a visual fullness indicating system.

The disposable wearable absorbent article 100C also includes another longitudinally oriented visual fullness indicator 135, along the longitudinal centerline 113 in the back 105. The back 105 also includes primary visual indicia represented by the letter A, secondary visual indicia represented by the letter B, and a tertiary visual indicium represented by the letter C. Together, the visual fullness indicator 135 along with the primary, secondary, and tertiary visual indicia A, B, and C can be considered a visual fullness indicating system. In alternate embodiments, the tertiary visual indicium C may not be present in the system.

In the disposable wearable absorbent article 100C, the indicators are oriented substantially radially out from the center 119. However, in addition to the locations and orientations illustrated in FIG. 1C, a visual fullness indicator of the present disclosure can be disposed in various alternate locations and orientations in an absorbent article, as will be understood by one of ordinary skill in the art. As an example, a visual fullness indicator can be disposed in a pant-type disposable wearable absorbent article in a lateral orientation or at an angle with respect to a centerline of the article.

FIG. 2A illustrates an outside perspective view of a front 201 and a side 203 of a front-fastenable disposable wearable absorbent article 200A formed for wearing. The absorbent article 200A includes a front waist edge 202 and a back waist edge 204. The pant-type disposable wearable absorbent article 200A also includes a waist opening 207 and a leg opening 208. The absorbent article 200A includes a longitudinally oriented visual fullness indicator 231 disposed in the front 201. The absorbent article 200A further includes primary visual indicia represented by the letter A, secondary visual indicia represented by the letter B, and a tertiary visual indicium represented by the letter C, disposed in the front 201. Together, the visual fullness indicator 231 along with the primary, secondary, and tertiary visual indicia A, B, and C can be considered a visual fullness indicating system.

While the present disclosure refers to front-fastenable absorbent articles, the present disclosure also contemplates alternate embodiments of absorbent articles having visual fullness indicators along with primary and secondary visual indicia, as described herein, wherein the absorbent articles are rear-fastenable. Thus, each embodiment of an absorbent article of the present disclosure that is described as front-fastenable can also be configured to be rear fastenable, as will be understood by one of ordinary skill in the art.

FIG. 2B illustrates an outside perspective view of a side 203 and a back 205 of a front-fastenable disposable wearable absorbent article 200B formed for wearing. The absorbent article 200B includes a front waist edge 202 and a back waist edge 204. The pant-type disposable wearable absorbent article 200B also includes a waist opening 207 and a leg opening 208. The absorbent article 200B includes a longitudinally oriented visual fullness indicator 235 disposed in the back 205. The absorbent article 200B further includes primary visual indicia represented by the letter A, secondary visual indicia represented by the letter B, and a tertiary visual indicium represented by the letter C, disposed in the front 201. Together, the visual fullness indicator 235 along with the primary, secondary, and tertiary visual indicia A, B, and C can be considered a visual fullness indicating system.

FIG. 2C illustrates an outside plan view of a front-fastenable disposable wearable absorbent article 200C laid out flat. The disposable wearable absorbent article 200C includes a front 201, a back 205, a longitudinal centerline 213, and a lateral centerline 216. The absorbent article 200C also includes a front waist edge 202 and a back waist edge 204.

The disposable wearable absorbent article 200C includes a topsheet 206, an outer cover 209, an acquisition layer 291, a distribution layer 293, and an absorbent core 295. A portion of the outer cover 209 is shown as broken to illustrate a portion of the topsheet 206 and a portion of the absorbent core 295. A portion of the absorbent core 295 is shown as broken to illustrate a portion of the distribution layer 293. A portion of the distribution layer 293 is shown as broken to illustrate a portion of the acquisition layer 291.

The disposable wearable absorbent article 200C includes a number of indicators in various exemplary locations and orientations. The disposable wearable absorbent article 200C includes a longitudinally oriented visual fullness indicator 231, along the longitudinal centerline 213 in the front 201. The front 201 also includes primary visual indicia represented by the letter A, secondary visual indicia represented by the letter B, and a tertiary visual indicium represented by the letter C. Together, the visual fullness indicator 231 along with the primary, secondary, and tertiary visual indicia A, B, and C can be considered a visual fullness indicating system.

The disposable wearable absorbent article 200C also includes a longitudinally oriented visual fullness indicator 235, along the longitudinal centerline 213 in the back 205. The back 205 also includes primary visual indicia represented by the letter A, secondary visual indicia represented by the letter B, and a tertiary visual indicium represented by the letter C. Together, the visual fullness indicator 235 and along with the primary, secondary, and tertiary visual indicia A, B, and C can be considered a visual fullness indicating system. In alternate embodiments, the tertiary visual indicium C may not be present in the system.

In the disposable wearable absorbent article 200C, the indicators are oriented substantially radially out from the center 219. However, in addition to the locations and orientations illustrated in FIG. 2C, a visual fullness indicator of the present disclosure can be disposed in various alternate locations and orientations in an absorbent article, as will be understood by one of ordinary skill in the art. As an example, a visual fullness indicator can be disposed in a front-fastenable disposable wearable absorbent article in a lateral orientation or at an angle with respect to a centerline of the article.

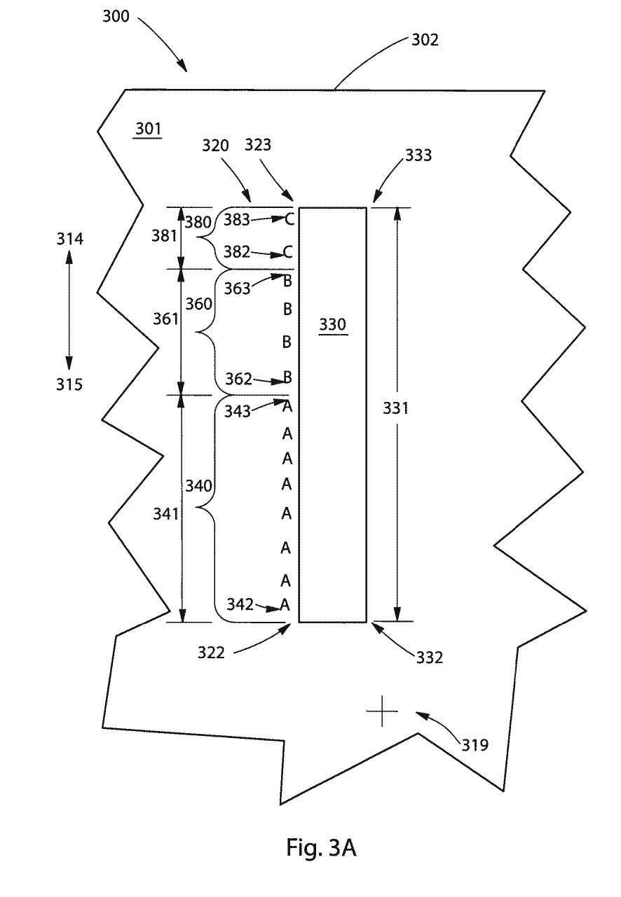

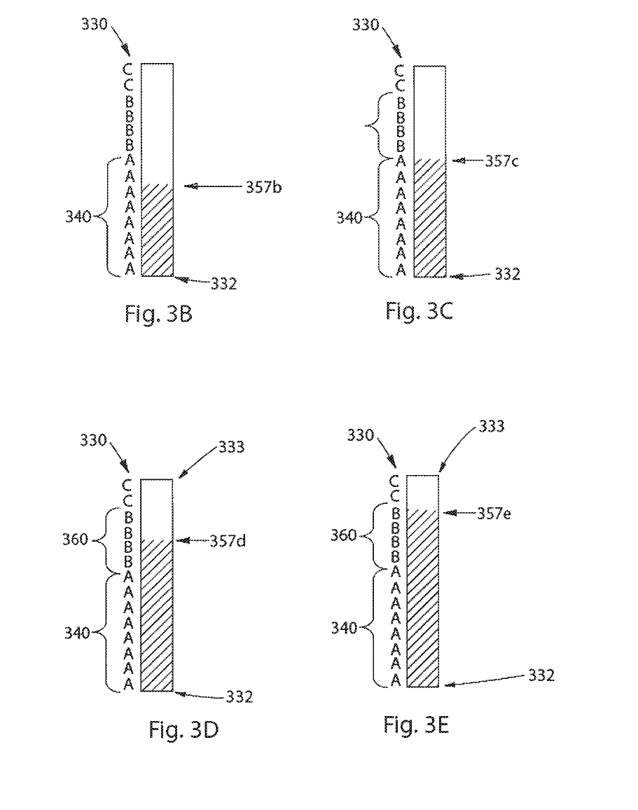

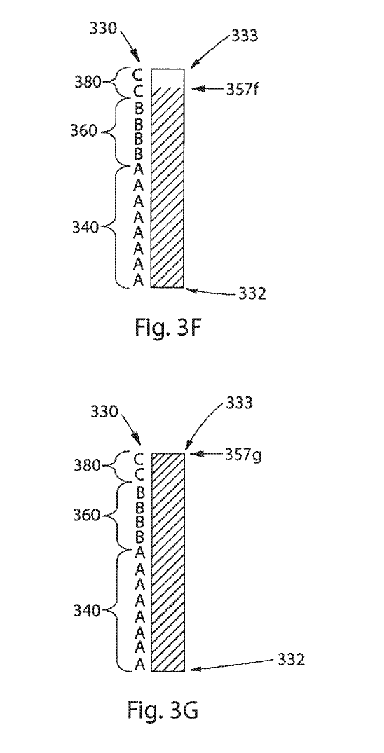

FIG. 3A illustrates an outside plan view of a portion of a front 301 of an absorbent article 300 laid out flat. In various embodiments, the absorbent article 300 can be a disposable wearable absorbent article, such as a pant-type disposable wearable absorbent article or a front-fastenable disposable wearable absorbent article. In FIG. 3A, the portion of the front 301 is bounded by a front waist edge 302 of the absorbent article 300 and by broken lines, since the portion is illustrated as separate from the rest of the absorbent article 300. For reference, FIG. 3A illustrates arrows indicating relative directions for longitudinally outboard 314 and longitudinally inboard 315 for the absorbent article 300. While the embodiment of FIG. 3A is illustrated with respect to the front 301, the embodiment can be similarly disposed in a back of an absorbent article.

The portion of the front 301 of the absorbent article 300 includes a wetness indicator 330. The wetness indicator 330 is a visual fullness indicator. However, in various embodiments the wetness indicator 330 can be configured as another form of visual wetness indicator, as will be understood by one of skill in the art.

The visual fullness indicator 330 is disposed offset from a center 319 of the absorbent article 300. In various embodiments, one or more parts of an indicator can be disposed near, at, or overlapping a center of an absorbent article. For example, a single indicating area can extend from a front of an absorbent article, through the center of the absorbent article, to the back of the absorbent article. In such an embodiment, a farthest inboard point along the indicating area can be considered an inboard end of two indicators.

The visual fullness indicator 330 includes one visual fullness indicating area. The visual fullness indicator 330 includes a longitudinally inboard end 332 and a longitudinally outboard end 333. The visual fullness indicator 330 has an overall indicator length 331, measured along the visual fullness indicator 330 from the inboard end 332 to the outboard end 333. The visual fullness indicator 330 has an overall shape that is substantially elongated and has a substantially uniform width along its entire overall indicator length.

In various embodiments an indicator and/or an indicating area can have an overall shape that is more or less elongated. In some embodiments, part, or parts, or all of an indicator and/or an indicating area can be straight, curved, angled, segmented, or any regular or irregular geometric shape (such as a square, rectangle, triangle, trapezoid, octagon, hexagon, star, half circle, a quarter circle, a half oval, a quarter oval, a radial pattern, etc.), a recognizable image (such as a letter, number, word, character, face of an animal, face of a person, etc.), or another recognizable image (such as a plant, a car, etc.), or another shape, or combinations of any of these shapes. Also, in various embodiments, an indicator and/or an indicating area can have varying widths over part, or parts, or all of its length.

A visual fullness indicator is a visually distinct and recognizable pathway of one or more visual indicators and/or visual indicating areas. A pathway is recognizable in its visual context. In other words, a pathway is distinct and recognizable, when compared with the appearance of a surrounding area. The pathway of a visual fullness indicator has two defined ends, a middle between the two ends, and a defined length from its one end to its other end. A visual fullness indicator can have one or more widths, each of which is less than its defined length.

A visual fullness indicator can be configured in various forms. For example, a visual fullness indicator can be formed by a single, continuous indicating area disposed along a pathway. As another example, a visual fullness indicator can be formed by a plurality of discrete indicators and/or discrete indicating areas disposed along a pathway.

The visual fullness indicator 330 is in fluid communication with an absorbent core of the absorbent article 300 along its entire overall indicator length. In various embodiments, a visual indicator can be configured such that part, or parts, or substantially all, or all of the indicator is in fluid communication with an absorbent core. In some embodiments, a visual indicator can be configured such that part, or parts, or substantially all, or all of the indicator overlaps an absorbent core or such that part, or parts, or substantially all, or all of the indicator does not overlap an absorbent core.

Throughout the present disclosure, fluid communication refers to a configured structural relationship that allows a liquid substance to freely pass from one element or location to another element or location; however, one element or location is not necessarily considered to be in fluid communication with another element or location merely by being connected or joined to a common element through which liquid can possibly pass. This definition of fluid communication is further explained by the following examples.

For example, if one element is configured to be in direct physical contact with another element such that a liquid substance can freely pass from the one element through the contacting portions to the other element, then the elements can be considered to be in fluid communication. As another example, if one element is connected to another element by a means for fluid communication such that a liquid substance can freely pass from the one element through the means for fluid communication to the other element, then the elements can be considered to be in fluid communication.

As a further example, if one element is connected to a substrate and another element is connected to the same substrate, but the substrate does not allow a liquid substance to freely pass through, then the elements are considered to be out of fluid communication. This holds true even if liquid can possibly pass through the substrate, so long as the liquid cannot pass through freely. The above definition of fluid communication, as explained through these examples, will be understood by one of ordinary skill in the art.

Throughout the present disclosure, the term liquid bodily exudate refers to any bodily substances exuded in liquid form (e.g. urine) and/or any liquid-like bodily substances (e.g. runny feces).

The primary visual fullness indicator 330 is configured to change from one or more initial visual states to one or more subsequent visual states when indicating the presence of a liquid bodily exudate. Throughout the present disclosure, the term visual state refers to an appearance which can be perceived by an unaided human with normal vision in standard lighting conditions. A visual state can comprise one or more colors, variations of color(s), patterns, letters, numbers, symbol, designs, images, and/or other visual devices. Colors include well known colors such as red, orange, yellow, green, blue, purple, etc. Variations of a color include variations in chroma, hue, and brightness, among others. While these informal terms are used for ease of reference, embodiments of the present disclosure are intended to encompass all colors which can be perceived by an unaided human with normal vision in standard lighting conditions.

In various embodiments, part, or parts, or all of a visual fullness indicating area can be configured to change from one or more initial visual states to one or more subsequent visual states. Also, in embodiments of the present disclosure, for a particular portion of a visual fullness indicating area, an initial visual state and a subsequent visual can each be any visual state, so long as the subsequent visual state is visually distinguishable from the first initial visual state.

Throughout the present disclosure, visual states are considered visually distinguishable if they can be recognized as different on sight by an unaided human with normal vision in standard lighting conditions. As an example, an unaided human with normal vision in standard lighting conditions should be able to recognize blue and yellow as different colors on sight. Thus, the blue and the yellow would be considered visually distinguishable visual states. As another example, an unaided human with normal vision in standard lighting conditions may be able to recognize a light shade of orange and a dark shade of orange as different shades of a color on sight. Thus, the light shade of orange and the dark shade of orange would be considered visually distinguishable visual states. As a further example, an unaided human with normal vision in standard lighting conditions may be able to recognize a first pattern and a second pattern as different visual states on sight. Thus, the first pattern and the second pattern would be considered visually distinguishable visual states.

As a still further example, an unaided human with normal vision in standard lighting conditions should be able to recognize an area with letters and a blank area as different visual states on sight. Thus, the area with letters and the blank area would be considered visually distinguishable visual states. Similarly, an area with numbers, symbols, designs, images, and/or other visual devices would also be considered visually distinguishable from a blank area or from a uniformly colored area. In addition to these examples, there are many other possible visually distinguishable visual states, as will be understood by one or ordinary skill in the art.

There are several ways by which absorbent articles of the present disclosure can be configured to include visual indicators that change visual states when indicating the presence of a bodily exudate, as will be understood by one of ordinary skill in the art. For example, an absorbent article can be configured to include such visual fullness indicators as described in the following U.S. Pat. Nos. 4,022,211, entitled “Wetness indicator for absorbent pads” issued on May 10, 1977 to Timmons, et al.; 4,231,370, entitled “Disposable diaper type garment having wetness indicator” issued on Nov. 4, 1980 to Mroz, et al.; 4,327,731, entitled “Moisture indicator” issued on May 4, 1982 to Powell; 4,681,576, entitled “Wetness indicating hot-melt adhesive” issued on Jul. 21, 1987 to Colon, et al.; 4,705,513, entitled “Disposable diaper with wetness indicator” issued on Nov. 10, 1987 to Sheldon, et al.; 4,738,674, entitled “Moisture indicator apparatus and method” issued on Apr. 19, 1988 to Todd, et al.; 4,743,238, entitled “Wetness indicating hot-melt adhesive” issued on May 10, 1988 to Colon et al.; 4,895,567, entitled “Wetness indicating hot-melt adhesive” issued on Jan. 23, 1990 to Colon et al.; 4,931,051, entitled “Wetness indicator” issued on Jun. 5, 1990 to Castello; 5,035,691, entitled “Hot melt moisture indicator material for disposable articles” issued on Jul. 30, 1991 to Zimmel, et al.; 5,066,711, entitled “Wetness indicating hot-melt adhesive” issued on Nov. 19, 1991 to Colon et al.; 5,089,548, entitled “Hot melt moisture indicator material for disposable articles” issued on Feb. 18, 1992 to Zimmel, et al.; 5,167,652, entitled “Moisture sensitive film” issued on Dec. 1, 1992 to Mueller; 5,342,861, entitled “Hot melt wetness indicator” issued on Aug. 30, 1994 to Raykovitz; 5,354,289 entitled “Absorbent product including super absorbent material and fluid absorption capacity monitor” issued on Oct. 11, 1994 to Mitchell, et al.; H1,376, entitled “Capacity visual indicia for absorbent articles” issued on Nov. 1, 1994 to Osborne, et al.; 5,647,863, entitled “Absorbent article with clean appearance and capacity signal means” issued on Jul. 15, 1997 to Hammons, et al.; 5,690,624, entitled “Disposable diaper” issued on Nov. 25, 1997 to Sasaki, et al.; 5,766,212, entitled “Disposable diaper” issued on Jun. 16, 1998 to Jitoe, et al,; 6,075,178, entitled “Absorbent article with wetness indicator” issued on Jun. 13, 2000; 6,515,194, entitled “Diaper having centrally-located chromatographic layer with peripherally-located wetness indicator” issued on Feb. 4, 2003 to Neading, et al.; 6,596,918, entitled “Absorbent articles having wetness indicating graphics and employing masking techniques” issued on Jul. 22, 2003 to Wehrle, et al.; 6,653,522, entitled “Hot melt adhesives based on sulfonated polyesters comprising wetness indicator” issued on Nov. 25, 2003 to Blumenthal, et al.; 6,772,708, entitled “Wetness indicator having improved colorant retention” issued on Aug. 10, 1994 to Klofta, et al.; 6,904,865, entitled “Wetness indicator having improved colorant retention and durability” issued on Jun. 14, 2005 to Klofta, et al.; 7,159,532, entitled “Wetness indicator having improved colorant retention and durability” issued on Jan. 9, 2007 to Klofta, et al.; 7,172,667, entitled “System and method for incorporating graphics into absorbent articles” issued on Feb. 6, 2007 to Vergona; 7,178,571, entitled “System and method for incorporating graphics into absorbent articles” issued on Feb. 20, 2007 to Vergona; 7,306,764, entitled “Wetness indicator” issued on Dec. 11, 2007 to Mody; and 7,332,642, entitled “Disposable absorbent articles having printed wetness indicators” issued on Feb. 19, 2008 to Liu, each of which is incorporated herein by reference.

The absorbent article 300 can be configured such that part, or parts, or all of the primary visual fullness indicator 330 is visible from outside of the absorbent article 300 when the absorbent article 300 is worn by a wearer. As a result, at least some of the subsequent visual state of the primary visual fullness indicator 330 will be visible from outside of the absorbent article 300.

A visual fullness indicating system with at least a primary visual indicium and a secondary visual indicium can help provide certainty about the fullness of the absorbent article 300. The portion of the front 301 of the absorbent article 300 includes visual indicia 320. The visual indicia 320 include a primary group 340 of primary visual indicia represented by the letter A, a secondary group 360 of secondary visual indicia represented by the letter B, and a tertiary group 380 of tertiary visual indicia represented by the letter C.

An visual indicium is visual device can be configured to visually indicate a particular degree of fullness by comprising one or more colors, variations of color(s), patterns, textures, letters, numbers, symbols, designs, images, shapes, and/or other visual devices, configured to indicate a degree of fullness. In some embodiments each visual indicium in a group of visual indicium can be its own distinct visual element, while in other embodiments, one or more visual indicium in a group of visual indicia can be represented together by a single visual element, as described herein.

In various embodiments, one visual indicium or multiple visual indicia can be represented by a shape that is straight, curved, angled, segmented, or any regular or irregular geometric shape (such as a square, rectangle, triangle, trapezoid, octagon, hexagon, star, half circle, a quarter circle, a half oval, a quarter oval, a radial pattern, etc.), a recognizable image (such as a letter, number, word, character, face of an animal, face of a person, etc.), or another recognizable image such as a plant, a car, etc.), or another shape, or combinations of any of these shapes.

A visual indicium can be any visual device, so long as a primary visual indicium is visually distinguishable from a secondary visual indicium, and a tertiary visual indicium is visually distinguishable for a secondary visual indicium. For example, a primary visual indicium can be green, a secondary visual indicium can be yellow, and a third visual indicium can be red.

The absorbent article 300 can be configured such that part, or parts, or all of the primary group 340, the secondary group 360, and the tertiary group 380 of indicia is visible from outside of the absorbent article 300 when the absorbent article 300 is worn by a wearer. As a result, at least some of the primary indicia A, the secondary indicia B, and the tertiary indicia C will be visible from outside of the absorbent article 300.

The primary group 340 includes a longitudinally inboard end 342, a longitudinally outboard end 343, and an overall primary group length 341, measured from the inboard end 342 to the outboard end 343. The secondary group 360 includes a longitudinally inboard end 362, a longitudinally outboard end 363, and an overall secondary group length 361, measured from the inboard end 362 to the outboard end 363. The tertiary group 380 includes a longitudinally inboard end 382, a longitudinally outboard end 383, and an overall tertiary group length 381, measured from the inboard end 382 to the outboard end 383. Where there is only one visual indicium, the farthest inboard point on the visual indicium is considered an inboard end and the farthest outboard point on the visual indicium is considered an outboard end.

Each of the primary visual indicia A is a configured to visually indicate a first degree of fullness. Each of the secondary visual indicia B is a configured to visually indicate a second degree of fullness. Each of the tertiary visual indicia C is a configured to visually indicate a third degree of fullness. In each group, each visual indicia is configured to have a similar visual appearance or the same visual appearance as the other visual indicia in that group.

While the portion of the front 301 of the absorbent article 300 includes eight primary visual indicia A, four secondary visual indicia B, and two tertiary visual indicia, an absorbent article can include various numbers of such visual indicia. The visual indicia A, B, and C are configured to correlate the progressive presence of the subsequent visual state in the visual fullness indicator 330 with varying degrees of fullness in the absorbent article 300.

The primary visual indicia A are configured to correlate with a first degree of fullness in the absorbent article 300. Since the secondary visual indicia B are disposed longitudinally outboard from the primary visual indicia A, the secondary visual indicia B are configured to correlate with a second degree of fullness that is greater than the first degree of fullness. Since the tertiary visual indicia C are disposed longitudinally outboard from the secondary visual indicia B, the tertiary visual indicia C are configured to correlate with a third degree of fullness that is greater than the second degree of fullness.

In the embodiment of FIG. 3A, the primary, secondary, and tertiary visual indicia A, B, and C are disposed proximate to the visual fullness indicator 330. However, in various embodiments, such visual indicia can be disposed remote from a visual fullness indicator, but can use a visual means, such as connecting lines, to associate the visual indicia with locations along the visual fullness indicator. In the embodiment of FIG. 3A, the primary, secondary, and tertiary visual indicia A, B, and C are disposed along one longitudinal side of the visual fullness indicator 330. However, in some embodiments, some of these visual indicia can be disposed on opposite sides of a visual fullness indicator.

In the embodiment of FIG. 3A, the primary, secondary, and tertiary visual indicia A, B, and C are regularly spaced apart from each other. However, in various embodiments such visual indicia can be spaced apart from each other with irregular spacing. In the embodiment of FIG. 3A, the overall primary group length 341 of the primary group 340 is greater than the overall secondary group length 361 of the secondary group 340, which is greater than the overall tertiary group length 381 of the tertiary group 380. However, this is not required and in some embodiments, the group lengths can vary.

The visual fullness indicator 330 can be configured to change visual states progressively, as illustrated with FIGS. 3B-3G. First, the visual fullness indicator 330 can begin to change from an initial visual state to a subsequent visual state in a portion adjacent to the primary group 340 of primary visual indicia when indicating the presence of a liquid bodily exudate to a first extent in an absorbent core of the absorbent article 300. Second, the visual fullness indicator 330 can begin to change from an initial visual state to a subsequent visual state in a portion adjacent to the secondary group 360 of secondary visual indicia when indicating the presence of a liquid bodily exudate to a second extent in the absorbent core of the absorbent article 300. Third, the visual fullness indicator 330 can begin to change from an initial visual state to a subsequent visual state in a portion adjacent to the tertiary group 380 of tertiary visual indicia when indicating the presence of a liquid bodily exudate to a third extent in the absorbent core of the absorbent article 300. The partial or complete absence or presence of the subsequent visual states proximate to the primary, secondary, and/or tertiary visual indicia can indicate the fullness of the absorbent article 300.

As a visual fullness indicator changes visual states it provides an indicating signal. The visual indicia provides additional indicating signals that are separate from and in addition to the indicating signal from the indicator. This combination of signals is easy to understand, when taken together. Thus, the combination of a visual fullness indicator along with at least a primary visual indicium and a secondary visual indicium can help provide certainty about the fullness of an absorbent article. In the embodiment of FIG. 3A, the combination of the visual fullness indicator 330 and each of the groups of visual indicia 340, 360, and 380, can help provide certainty about the fullness of an absorbent article 300.

By knowing the fullness of an absorbent article, the absorbent article can be changed after a wearer has appropriately utilized its capacity and/or before it is likely to leak. Throughout the present disclosure, the term “capacity” is used to indicate the capacity of the absorbent core alone and does not include the absorbency of other components or structures, such as acquisition or distribution layers. The benefits of the combination of a visual fullness indicator along with primary and secondary visual indicia are similarly provided in the embodiments of FIGS. 3B-3G, as described below.

FIGS. 3B-3G illustrate the visual fullness indicator 330 along with the primary group 340, secondary group 360, and tertiary group 380 of visual indicia of the embodiment of FIG. 3A in various states of indication, wherein the indicator changes visual states progressively in the presence of a liquid bodily exudate to indicate the fullness of the absorbent article 300. In FIGS. 3B-3G, subsequent visual states are illustrated with hatch patterns.

FIG. 3B illustrates a subsequent state of indication for the indicator of FIG. 3A, wherein part of the visual fullness indicator 330 has changed from an initial visual state to a subsequent visual state, to indicate the fullness of the absorbent article 300. In FIG. 3B, a liquid bodily exudate has passed through a portion of the absorbent core of the absorbent article 300 in sufficient concentration to cause a change in visual state from the inboard end 332 up through part of the of the visual fullness indicator 330 to a wet edge 357B. The wet edge 357B is proximate to the middle of the primary group 340 of the primary visual indicia A.

Throughout the present disclosure, a wet edge refers to a boundary along a visual wetness indicator of an absorbent article, wherein the boundary indicates an extent of the presence of a liquid bodily exudate. On the inboard side of the wet edge, the visual wetness indicator has experienced the presence of a liquid bodily exudate at a concentration that is sufficient to cause the visual wetness indicator to change visual states. On the outboard side of the wet edge, the visual wetness indicator has not yet experienced the presence of a liquid bodily exudate at a concentration that is sufficient to cause the visual wetness indicator to change visual states.

FIG. 3C illustrates a subsequent state of indication for the indicator of FIG. 3B, wherein part of the visual fullness indicator 330 has changed from an initial visual state to a subsequent visual state, to indicate the fullness of the absorbent article 300. In FIG. 3C, a liquid bodily exudate has passed through a portion of the absorbent core of the absorbent article 300 in sufficient concentration to cause a change in visual state from the inboard end 332 up through part of the of the primary visual fullness indicator 330 to a wet edge 357C. The wet edge 357C is proximate to the outboard end of the primary group 340 of primary visual indicia A and to the inboard end of the secondary group 360 of the secondary visual indicia B.

FIG. 3D illustrates a subsequent state of indication for the indicator of FIG. 3C, wherein part of the visual fullness indicator 330 has changed from an initial visual state to a subsequent visual state, to indicate the fullness of the absorbent article 300. In FIG. 3D, a liquid bodily exudate has passed through a portion of the absorbent core of the absorbent article 300 in sufficient concentration to cause a change in visual state from the inboard end 332 up through part of the of the primary visual fullness indicator 330 to a wet edge 357D. The wet edge 357D is proximate to the middle of the secondary group 360 of the secondary visual indicia B.

FIG. 3E illustrates a subsequent state of indication for the indicator of FIG. 3D, wherein part of the visual fullness indicator 330 has changed from an initial visual state to a subsequent visual state, to indicate the fullness of the absorbent article 300. In FIG. 3E, a liquid bodily exudate has passed through a portion of the absorbent core of the absorbent article 300 in sufficient concentration to cause a change in visual state from the inboard end 332 up through part of the of the primary visual fullness indicator 330 to a wet edge 357E. The wet edge 357E is proximate to the outboard end of the secondary group 360 of the secondary visual indicia B and to the inboard end of the tertiary group 380 of the tertiary visual indicia C.

FIG. 3F illustrates a subsequent state of indication for the indicator of FIG. 3E, wherein part of the visual fullness indicator 330 has changed from an initial visual state to a subsequent visual state, to indicate the fullness of the absorbent article 300. In FIG. 3F, a liquid bodily exudate has passed through a portion of the absorbent core of the absorbent article 300 in sufficient concentration to cause a change in visual state from the inboard end 332 up through part of the of the primary visual fullness indicator 330 to a wet edge 357F. The wet edge 357F is proximate to the middle of the tertiary group 380 of the tertiary visual indicia C.

FIG. 3G illustrates a subsequent state of indication for the indicator of FIG. 3F, wherein all of the visual fullness indicator 330 has changed from an initial visual state to a subsequent visual state, to indicate the fullness of the absorbent article 300. In FIG. 3G, a liquid bodily exudate has passed through a portion of the absorbent core of the absorbent article 300 in sufficient concentration to cause a change in visual state from the inboard end 332 up through part of the of the primary visual fullness indicator 330 to a wet edge 357G. The wet edge 357G is proximate to the outboard end of the tertiary group 380 of the tertiary visual indicia C and to the outboard end 333 of the indicator 330.

Together, FIGS. 3B-3G illustrate that the indicator 330 can change visual states progressively in the presence of a liquid bodily exudate and, along with primary, secondary, and tertiary visual indicia, can indicate the degree to which a liquid bodily exudate has filled the absorbent article 300. In addition to indicating fullness, in embodiments of the present disclosure, such changes in visual state in a visual fullness indicating system can also be understood as a signal that indicates the remaining absorbent capacity of an absorbent article and/or as a signal that indicates the risk that an absorbent article may leak.

An appropriate particular location and orientation as well as specific dimensions and other physical characteristics for an indicator and/or visual indicia of the present disclosure can be selected in order for the indicator and/or visual indicia to provide signals that indicate the degree of fullness, the remaining capacity, and/or the leakage risk for a particular absorbent article. For each group of visual indicia of the present disclosure, the location of the inboard end and the outboard end can be selected to provide visual signals that indicate the degree of fullness, the remaining capacity, and/or the leakage risk for the absorbent article in which a visual fullness indicator is included. The degree of fullness, the remaining capacity, and/or the leakage risk for a particular absorbent article can be determined as described in US non-provisional patent application entitled “Absorbent Articles with Primary and Secondary Indicating,” filed on Dec. 23, 2009 under attorney docket number 11222MQ, which is incorporated herein by reference.

As a first example, in various embodiments, an inboard end of a primary group of visual indicia can be disposed at a particular location, such that, a change in visual state proximate to or at that inboard end (i.e. a visual fullness indicator with a wet edge proximate to that inboard end) indicates that the absorbent article has: (a) a fullness of about 25%, 30%, 35%, 40%, 45%, 50%, 55%, 60%, 65%, 70%, 75%, 80%, 85%, or any integer of percentage between any of these values, or within any range using any of these values; (b) a remaining capacity of about 75%, 70%, 65%, 60%, 55%, 50%, 45%, 40%, 35%, 30%, 25%, 20%, 15%, or any integer of percentage between any of these values, or within any range using any of these values; and/or (c) a leakage risk of >0%, 5%, 10%, 15%, 20%, 25%, 30%, 35%, 40%, 45%, 50%, 55%, 60%, 65%, 70%, 75%, 80%, 85%, or any integer of percentage between any of these values, or within any range using any of these values. As used herein, the term “leakage risk” refers to the probability of a liquid bodily exudate leaking out of a diaper, while the diaper is being properly worn by a wearer of appropriate size, with such probability being measured in a sufficient number of diapers being used by a sufficient number of wearers of appropriate size. For example, at least 100 users should use at least five days worth of diapers to determine the probability of a diaper leaking.

An inboard end of a primary group of visual indicia can be disposed in an absorbent article at any of the following locations: 90 mm, 80 mm, 70 mm, 60 mm, 50 mm, 40 mm, 30 mm, 20 mm, 10 mm, or 5 mm inboard to an outer edge of an acquisition layer; at an outer edge of an acquisition layer; 80 mm, 70 mm, 60 mm, 50 mm, 40 mm, 35 mm, 30 mm, 25 mm, 20 mm, 15 mm, 10 mm, or 5 mm inboard to an outer edge of a distribution layer; at an outer edge of a distribution layer; 140 mm, 130 mm, 120 mm, 110 mm, 100 mm, 90 mm, 80 mm, 70 mm, 60 mm, 50 mm, 40 mm, 30 mm, 20 mm, 10 mm, or 5 mm inboard to an outer edge of an absorbent core; or at an outer edge of an absorbent core. An inboard end of a primary group of visual indicia can also be disposed in an absorbent article at any integer of mm between any of these values or within any range using any of these values. Where there is only one primary visual indicium, the primary visual indicium can be disposed at any of the locations for an inboard end of a primary group of visual indicia.

As a second example, in various embodiments, an outboard end of a primary group of visual indicia can be disposed at a particular location, such that, a change in visual state proximate to or at that outboard end (i.e. a visual fullness indicator with a wet edge proximate to that outboard end) indicates that the absorbent article has: (a) a fullness of 30%, 35%, 40%, 45%, 50%, 55%, 60%, 65%, 70%, 75%, 80%, 85%, 90%, or any integer of percentage between any of these values, or within any range using any of these values; (b) a remaining capacity of 70%, 65%, 60%, 55%, 50%, 45%, 40%, 35%, 30%, 25%, 20%, 15%, 10%, or any integer of percentage between any of these values, or within any range using any of these values; and/or (c) a leakage risk of 30%, 35%, 40%, 45%, 50%, 55%, 60%, 65%, 70%, 75%, 80%, 85%, 90%, or any integer of percentage between any of these values, or within any range using any of these values.

An outboard end of a primary group of visual indicia can be disposed in an absorbent article at any of the following locations: 80 mm, 70 mm, 60 mm, 50 mm, 40 mm, 30 mm, 20 mm, 10 mm, or 5 mm inboard to an outer edge of an acquisition layer; at an outer edge of an acquisition layer; 5 mm or 10 mm outboard from an outer edge of an acquisition layer; 70 mm, 60 mm, 50 mm, 40 mm, 30 mm, 25 mm, 20 mm, 15 mm, 10 mm, or 5 mm inboard to an outer edge of a distribution layer; at an outer edge of a distribution layer; 5 mm or 10 mm outboard from an outer edge of a distribution layer; 130 mm, 120 mm, 110 mm, 100 mm, 90 mm, 80 mm, 70 mm, 60 mm, 50 mm, 40 mm, 30 mm, 20 mm, 10 mm, or 5 mm inboard to an outer edge of an absorbent core; at an outer edge of an absorbent core; or 5 mm or 10 mm outboard from an outer edge of an absorbent core. An outboard end of a primary group of visual indicia can also be disposed in an absorbent article at any integer of mm between any of these values, or within any range using any of these values. Where there is only one primary visual indicium, the primary visual indicium can be disposed at any of the locations for an outboard end of a primary group of visual indicia.

As a third example, in various embodiments, an inboard end of a secondary group of visual indicia can be disposed at a particular location, such that, a change in visual state at that inboard end (i.e. a visual fullness indicator with wet edge proximate to that inboard end) indicates that the absorbent article has: (a) a fullness of 50%, 55%, 60%, 65%, 70%, 75%, 80%, 85%, 90%, 95% or any integer of percentage between any of these values, or within any range using any of these values; (b) a remaining capacity of 50%, 45%, 40%, 35%, 30%, 25%, 20%, 15%, 10%, 5%, or any integer of percentage between any of these values, or within any range using any of these values; and/or (c) a leakage risk of 10%, 20%, 30%, 40%, 50%, 60%, 70%, 80%, 90%, or any integer of percentage between any of these values, or within any range using any of these values

An inboard end of a secondary group of visual indicia can be disposed in an absorbent article at any of the following locations: 75 mm, 70 mm, 60 mm, 50 mm, 40 mm, 30 mm, 20 mm, 10 mm, or 5 mm inboard to an outer edge of an acquisition layer; at an outer edge of an acquisition layer; or 5 mm, 10 mm, or 20 mm outboard from an outer edge of an acquisition layer; 75 mm, 70 mm, 660 mm, 50 mm, 40 mm, 30 mm, 25 mm, 20 mm, 15 mm, 10 mm, or 5 mm inboard to an outer edge of a distribution layer; at an outer edge of a distribution layer; or 5 mm, 10 mm, or 20 mm outboard from an outer edge of a distribution layer; 120 mm, 110 mm, 100 mm, 90 mm, 80 mm, 70 mm, 60 mm, 50 mm, 40 mm, 30 mm, 20 mm, or 10 mm inboard to an outer edge of an absorbent core; at an outer edge of an absorbent core; or 5 mm, 10 mm, or 20 mm outboard from an outer edge of an absorbent core. An inboard end of a secondary visual wetness indicator can also be disposed in an absorbent article at any integer of mm between any of these values, or within any range using any of these values.

In various embodiments, a secondary group of visual indicia disposed in the back of an absorbent article can have an inboard end disposed with respect to a longitudinally outboard edge of an absorbent core disposed in the front of the article. In this way, the indicating signal from the secondary group of visual indicia can be configured with respect to a point in the front/center of the article, where liquid bodily exudates are provided to the article by the wearer. As examples, a secondary group of visual indicia can be disposed in the back of an article with an inboard end of the group disposed 275 mm, 270 mm, 260 mm, 250 mm, 240 mm, 230 mm, 220 mm, 210 mm, 200 mm, 190 mm, 180 mm, 170 mm, 160 mm, or 150 mm, from a longitudinally outboard edge of an absorbent core disposed in the front of the article. An inboard end of a secondary group of visual indicia can also be disposed in an absorbent article at any integer of mm between any of these values, or within any range using any of these values. Where there is only one secondary visual indicium, the secondary visual indicium can be disposed at any of the locations for an inboard end of a secondary group of visual indicia.

A change in visual state proximate to or at an inboard end of a secondary group of visual indicia may indicate a fullness that is less than, or equal to, or greater than a fullness indicated by a change in visual state proximate to or at an outboard end of a primary group of visual indicia. A change in visual state proximate to or at an inboard end of a secondary group of visual indicia may indicate a remaining capacity that is greater than, or equal to, or less than a remaining capacity indicated by a change in visual state proximate to or at an outboard end of a primary group of visual indicia. A change in visual state proximate to or at an inboard end of a secondary group of visual indicia may indicate a leakage risk that is greater than, or equal to, or less than a leakage risk indicated by a change in visual state proximate to or at an outboard end of a primary group of visual indicia.

As a fourth example, in various embodiments, an outboard end of a secondary group of visual indicia can be disposed at a particular location, such that, a change in visual state at that outboard end (i.e. a visual fullness indicator with wet edge proximate to that outboard end) indicates that the absorbent article has: (a) a fullness of 55%, 60%, 65%, 70%, 75%, 80%, 85%, 90%, 95%, about 100%, or any integer of percentage between any of these values, or within any range using any of these values; (b) a remaining capacity of 45%, 40%, 35%, 30%, 25%, 20%, 15%, 10%, 5%, about 0%, or any integer of percentage between any of these values, or within any range using any of these values; and/or (c) a leakage risk of 20%, 30%, 40%, 50%, 60%, 70%, 80%, 90%, about 100%, or any integer of percentage between any of these values, or within any range using any of these values. Where there is only one secondary visual indicium, the secondary visual indicium can be disposed at any of the locations for an outboard end of a secondary group of visual indicia.

An outboard end of a secondary group of visual indicia can be disposed in an absorbent article at any of the following locations: 50 mm, 40 mm, 30 mm, 20 mm, 10 mm, or 5 mm inboard to an outer edge of an acquisition layer; at an outer edge of an acquisition layer; 5 mm, 10 mm, 20 mm, or 30 mm outboard from an outer edge of an acquisition layer; 50 mm, 40 mm, 30 mm, 25 mm, 20 mm, 15 mm, 10 mm, or 5 mm inboard to an outer edge of a distribution layer; at an outer edge of a distribution layer; 5 mm, 10 mm, 20 mm, or 30 mm outboard from an outer edge of a distribution layer; 110 mm, 100 mm, 90 mm, 80 mm, 70 mm, 60 mm, 50 mm, 40 mm, 30 mm, 20 mm, or 10 mm inboard to an outer edge of an absorbent core; at an outer edge of an absorbent core; or 5 mm, 10 mm, 20 mm, or 30 mm outboard from an outer edge of an absorbent core. An outboard end of a secondary group of visual indicia can also be disposed in an absorbent article at any integer of mm between any of these values, or within any range using any of these values. Where there is only one secondary visual indicium, the secondary visual indicium can be disposed at any of the locations for an inboard end of a secondary group of visual indicia.

It is contemplated that any of the four exemplary embodiments described above can be applied in any workable combination to any relevant embodiment of the present disclosure.

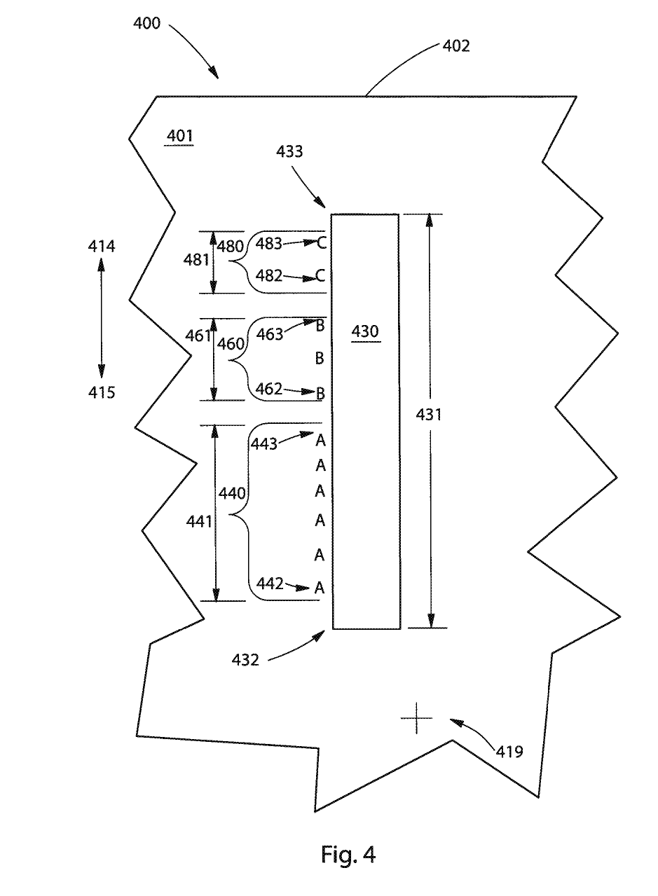

FIG. 4 illustrates an outside plan view of a portion of a front 401 of an absorbent article 400 laid out flat. In various embodiments, the absorbent article 400 can be a disposable wearable absorbent article, such as a pant-type disposable wearable absorbent article or a front-fastenable disposable wearable absorbent article. In FIG. 4, the portion of the front 401 is bounded by a front waist edge 402 of the absorbent article 400 and by broken lines, since the portion is illustrated as separate from the rest of the absorbent article 400. For reference, FIG. 4A illustrates arrows indicating relative directions for longitudinally outboard 414 and longitudinally inboard 415 for the absorbent article 400. While the embodiment of FIG. 4 is illustrated with respect to the front 401, the embodiment can be similarly disposed in a back of an absorbent article. Each of the elements of the embodiment of FIG. 4 is configured in the same way as the like-numbered element of the embodiment of FIG. 3A, except as noted below.

Throughout the present disclosure, the term “like-numbered” is intended to indicate a correspondence between labels of elements wherein the last two numbers in the labels of the elements are the same. Element labels are considered to be like-numbered despite differing numeral prefixes corresponding to figure numbers, and despite differing alphabetical suffixes corresponding to particular embodiments.

In the embodiment of FIG. 4, the inboard end 442 of the primary group 440 of primary visual indicia A is spaced apart from the inboard end 432 of the visual fullness indicator 430. The overall primary group length 441 is shorter than the overall primary group length 341 of the embodiment of FIG. 3A. The primary group 440 includes six primary visual indicia A.

The inboard end 462 of the secondary group 460 of secondary visual indicia B is spaced apart from the outboard end 443 of the primary group 440. The overall secondary group length 461 is shorter than the overall secondary group length 361 of the embodiment of FIG. 3A. The secondary group 460 includes three primary visual indicia B.

The inboard end 482 of the tertiary group 480 of tertiary visual indicia C is spaced apart from the outboard end 463 of the secondary group 460. The outboard end 433 of the visual fullness indicator 430 is spaced apart from the outboard end 483 of the tertiary group 480.

Further, the present disclosure contemplates that an absorbent article, such as a disposable wearable absorbent article, can have one or more visual fullness indicators configured as described herein and further configured with various additional and/or alternate structures and/or functions as described below.

One or more embodiments of the present disclosure can be combined with one or more embodiments of U.S. non-provisional patent application Ser. No. 12/346,445 entitled “Disposable Wearable Absorbent Articles with Multiple Indicating Colors,” filed on Dec. 30, 2008 under attorney docket number 11217Q and/or US non-provisional patent application entitled “Disposable Wearable Absorbent Articles with Multiple Indicating Colors,” filed on Dec. 23, 2009 and further identified by attorney docket number 11217RQ, each of which is incorporated herein by reference. A disposable wearable absorbent article with primary and secondary indicia can also have multiple indicating colors.

One or more embodiments of the present disclosure can be combined with one or more embodiments of U.S. non-provisional patent application Ser. No. 12/346,481 entitled “Absorbent Articles with Multiple Indicating Widths,” filed on Dec. 30, 2008 under attorney docket number 11218Q and/or US non-provisional patent application entitled “Absorbent Articles with Multiple Indicating Widths,” filed on Dec. 23, 2009 under attorney docket number 11218RQ, each of which is incorporated herein by reference. A disposable wearable absorbent article with primary and secondary indicia can also have multiple indicating widths.

One or more embodiments of the present disclosure can be combined with one or more embodiments of U.S. non-provisional patent application Ser. No. 12/346,496 entitled “Disposable Wearable Absorbent Articles with Gender Specific Indicia,” filed on Dec. 30, 2008 under attorney docket number 11219 and/or US non-provisional patent application entitled “Disposable Wearable Absorbent Articles with Gender Specific Indicia,” filed on Dec. 23, 2009 under attorney docket number 11219R, each of which is incorporated herein by reference. A disposable wearable absorbent article with primary and secondary indicia can also have gender specific indicia.

One or more embodiments of the present disclosure can be combined with one or more embodiments of U.S. non-provisional patent application Ser. No. 12/346,510 entitled “Disposable Wearable Absorbent Articles with Gender Specific Indicating,” filed on Dec. 30, 2008 under attorney docket number 11220 and/or US non-provisional patent application entitled “Disposable Wearable Absorbent Articles with Gender Specific Indicating,” filed on Dec. 23, 2009 and further identified by attorney docket number 11220R, each of which is incorporated herein by reference. A disposable wearable absorbent article with primary and secondary indicia can also have gender specific indicating.

One or more embodiments of the present disclosure can be combined with one or more embodiments of U.S. non-provisional patent application Ser. No. 12/346,520 entitled “Absorbent Articles with Patterns of Indicating,” filed on Dec. 30, 2008 under attorney docket number 11221Q and/or US non-provisional patent application entitled “Absorbent Articles with Patterns of Indicating,” filed on Dec. 23, 2009 under attorney docket number 11221RQ, each of which is incorporated herein by reference. A disposable wearable absorbent article with primary and secondary indicia can also have patterns of indicating.

One or more embodiments of the present disclosure can be combined with one or more embodiments of U.S. provisional patent application 61/141,573 entitled “Absorbent Articles with Primary and Secondary Indicating,” filed on Dec. 30, 2008 under attorney docket number 11222PQ and/or US non-provisional patent application entitled “Absorbent Articles with Primary and Secondary Indicating,” filed on Dec. 23, 2009 under attorney docket number 11222MQ, each of which is incorporated herein by reference. A disposable wearable absorbent article with primary and secondary indicia can also have primary and secondary indicating.

An absorbent article having primary visual indicia and secondary visual indicia along with a visual fullness indicator can help provide certainty about the fullness of the absorbent article. By knowing how full an article is, the article can be changed after the wearer has appropriately utilized the capacity of the article. Also, by knowing how full an article is, the article can be changed before it is likely to leak.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article, comprising:
a visual fullness indicator;
a primary group of primary visual indicia disposed proximate to the visual fullness indicator and configured to visually indicate a first degree of fullness in the article; and
a secondary visual indicium that is visually distinguishable from the primary visual indicia, disposed proximate to the visual fullness indicator, and configured to indicate a second degree of fullness in the article that is greater than the first degree of fullness.

2. The absorbent article of claim 1, which is a disposable absorbent article.

3. The disposable absorbent article of claim 2, which is a disposable wearable absorbent article.

4. The absorbent article of claim 3, wherein:
the article includes an acquisition layer with an outboard end; and
a portion of the secondary visual indicium is disposed inboard to the outboard end of the acquisition layer.

5. The absorbent article of claim 4, wherein substantially all of the secondary visual indicium is disposed outboard from the outboard end of the acquisition layer.

6. The absorbent article of claim 5, wherein substantially all of the primary group is disposed inboard to the outboard end of the acquisition layer.

7. The absorbent article of claim 3, wherein:
the article includes a distribution layer with an outboard end; and
a portion of the secondary visual indicium is disposed inboard to the outboard end of the acquisition layer.

8. The absorbent article of claim 7, wherein substantially all of the secondary visual indicium is disposed outboard from the outboard end of the distribution layer.

9. The absorbent article of claim 8, wherein substantially all of the primary group is disposed inboard to the outboard end of the distribution layer.

10. The absorbent article of claim 3, wherein:
the article includes an absorbent core with an outboard end; and
a portion of the secondary visual indicium is disposed inboard to the outboard end of the absorbent core.

11. The absorbent article of claim 10, wherein substantially all of the secondary visual indicium is disposed outboard from the outboard end of the absorbent core.

12. The absorbent article of claim 11, wherein substantially all of the primary group is disposed inboard to the outboard end of the absorbent core.

13. The absorbent article of claim 3, wherein an overall length of the primary group is greater than an overall length of the secondary visual indicium.

14. The absorbent article of claim 3, wherein:
the article includes a secondary group of secondary visual indicia disposed proximate to the visual fullness indicator and configured to visually indicate the second degree of fullness in the article; and
the secondary group includes the secondary visual indicium.

15. The absorbent article of claim 3, including a tertiary indicium disposed proximate to the visual fullness indicator and configured to visually indicate a third degree of fullness in the article.

16. An absorbent article, comprising:
an absorbent core with a total absorbent capacity;
a visual fullness indicator; and
a primary group of primary visual indicia disposed proximate to the visual fullness indicator;
a secondary visual indicium that is visually distinguishable from the primary visual indicia;
wherein the secondary visual indicium is disposed proximate to a particular location, a portion of the absorbent core is disposed longitudinally outboard from the particular location, and the portion of the absorbent core has an absorbent capacity that is less than or equal to about 25% of the total absorbent capacity.

17. The absorbent article of claim 16, wherein the portion of the absorbent core has an absorbent capacity that is less than or equal to about 20% of the total absorbent capacity.

18. The absorbent article of claim 17, wherein the portion of the absorbent core has an absorbent capacity that is less than or equal to about 15% of the total absorbent capacity.

19. An absorbent article, comprising:
an absorbent core with a total absorbent capacity;
a visual fullness indicator that is configured to change to a subsequent visual state when indicating the presence of at least a particular concentration of a bodily exudate;
a primary group of primary visual indicia disposed proximate to the visual fullness indicator;
a secondary visual indicium that is visually distinguishable from the primary visual indicia;
wherein the article is configured such that, when the absorbent core is loaded by a wearer of the article with a liquid bodily exudate to about 90% of the total absorbent capacity, the particular concentration of the liquid bodily exudate extends to a first particular location, and an inboard end of the secondary visual indicium is disposed inboard to the first particular location.

20. The absorbent article of claim 19, wherein the article is configured such that, when the absorbent core is loaded by a wearer of the article with a liquid bodily exudate to about 85% of the total absorbent capacity, the particular concentration of the liquid bodily exudate extends to a second particular location, and the inboard end of the secondary visual indicium is disposed inboard to the second particular location.
